(19) United States
(12) Patent Application Publication
(10) Pub. No.: US 2010/0160843 A1
(43) Pub. Date: Jun. 24, 2010

(54) Inflatable cast for treating a patient and method of making cast

(76) Inventor: Todd Neely, Green Oaks, IL (US)

Correspondence Address: MARSHALL A. BURMEISTER, P. O. BOX 765, WILLIAMS BAY, WI, 53191, US

(21) Appl. No.: 12/317,331

(22) Filed: Dec. 22, 2008

(51) Int. Cl. A61F 5/00

(52) U.S. Cl. 602/13; 602/28

(57) ABSTRACT

A cast comprising a shell of bendable material with opposed ends and opposed edges, the shell being bent into a conical shape that is adapted to surround and form a gap with a part of a patient. The edges are fastened on and sealed to each other, and the cast includes a layer of casting material about the shell. A stretchable membrane is disposed in the gap and sealed on the shell forming a fluid tight bag which when inflated engages the patient's part. Metal particles on the membrane surface opposite the shell produce an oligodynamic effect.

This invention relates generally to a cast for immobilizing a part of the body of either a human being or an animal in order to apply treatment to that part of the body. More particularly, the present invention relates to an inflatable cast that fits better and is more comfortable for the patient than conventional casts, and further permits the immobilized part of the patient to be examined, ventilated, medicated and sterilized without removing the cast from the patient. A cast according to the present invention is particularly useful in the treatment of broken bones, sprains, torn ligaments, and diabetic ulcers of the foot. The present invention also relates to methods of making such casts.

### BACKGROUND OF THE INVENTION

Typically, casts are constructed from a gauze material wrapped around the part of the patient being treated, and the gauze material is in turn covered with a rigid material, such as Plaster of Paris or fiberglass. The conventional procedure for making a cast is: (1) treat the injury, then cover any open wound; (2) wrap the part of the body to be immobilized and protected with cotton or synthetic padding; (3) cover the part of the body to be immobilized with a cotton or synthetic stockinette; (4) wrap casting material, usually fiberglass or Plaster of Paris, around the part of the body to be immobilized (5) wait for the casting material to dry; and (6) inspect the cast for a good fit. Such orthopedic casts support and protect injured bones and soft tissue, reduce pain, swelling and muscle spasms, but severely limit the ability of a patient's physician to examine the part of the patient's body within the cast, or to treat that part of the patient, and also provide a significant risk of complications as well as discomfort to the patient.

Patients using such orthopedic casts often experience cutaneous complications including macerations, ulcerations, infections, rashes, itching, burns and allergic contact dermatitis. Since the body area covered by such casts cannot be cleaned, pathogens under the cast present a high risk of serious infection to a patient with an open wound under the cast. Such patients are also at risk to staphylococcal infection of the hair follicles and sweat glands which results in severe and painful dermatitis. The body area covered by a typical cast also becomes dry and scaly, and the skin's naturally discarded epithelium cannot be readily removed resulting in unpleasant odors, annoying itching and general discomfort to the patient.

If a patient needs to bathe or swim, the typical cast cannot be used. A cast can be constructed using a waterproof breathable fabric available from W. L. Gore and Associates under the trademark PROCEL in place of the layer of gauze and enclosing the PROCEL layer with a rigid layer of waterproof fiberglass tape. In order to dry such a waterproof cast after it has been immersed in water, a prolonged procedure must be employed. First, as much water as possible must be drained from the cast. The remaining water is then converted into water vapor by the heat of the patient, and circulation of air through the cast removes the water vapor, a process that can take in excess of two hours and may leave soap and debris within the cast. Casts of this construction have increased patient comfort, but their use is limited as they cannot be used with an open wound.

An example of a treatment that may not be administered to the part of a patient that is immobilized within a conventional cast is sterilization. In the treatment of diabetic foot ulcers, the ulcerated foot of the patient is generally placed in a cast to limit the wound from pressure and protect it from injury while healing. However, it is also often desirable to treat the ulcerated foot against infection, and at present such treatment is limited by the inability to access the wound without removing the ulcerated foot from the cast. Examples of equipment requiring removal of the cast are disclosed in U.S. Pat. No. 6,800,064 to Liang, U.S. Pat. No. 6,872,366 to Thomas and U.S. Pat. No. 5,098,415 to Levin, which treat diabetic foot ulcers by subjecting the ulcerated foot to ozone to disinfect the wound, generally by placing the patient's foot in a chamber and circulating ozone through the chamber.

Many specialized devices have been developed in the past to provide added comfort to the patient or overcome specific problems in the treatment of injuries or diseases to the patient. U.S. Pat. No. 3,580,248 to Larson discloses a device having a rigid shell that conforms generally to the part of the patient's body being treated, the shell being divided into two mating portions and each portion being provided with an inflatable liner mounted within the shell. When in use, the two parts of the shell are secured together by brackets and the liners are inflated and then compressed between the part of the patient being treated and the shell. The Larson device may be removed from the patient at any time for observation or treatment of the immobilized part of the patient by deflating the liners and releasing the brackets, and thereafter repositioning the shell on the patient. U.S. Pat. No. 4,621,624 to Rayboy discloses a device which is provided with a bladder disposed between an immobilized part of a patient and a cast to improve the fit of the cast to the patient and to permit the bladder inflation to be adjusted if and when the patient's immobilized part shrinks in order to maintain the desired pressure on that part of the patient. Further, U.S. Pat. No. 5,288,286 to Davis discloses a device which is provided with a plurality of inflatable bladders disposed between an immobilized part of a patient and an external sleeve member constructed of non-rigid elastomeric material. Also, the Rayboy patent and U.S. Pat. No. 6,120,469 to Bruder disclose casts with ventilating systems communicating with air pumps in order to make the patient more comfortable.

Nonetheless, the health care industry makes little use of improvements to the casts known to the art to comfort, observe or treat the part of a patient within a cast. Some improvements facilitate removal of the cast from the patient to permit observation or treatment, but it is generally considered undesirable to remove an immobilized part of the patient from the cast because of the possibility of injury to the patient. Other improvements replace the typical cast with lighter or flexible materials, but it is generally considered desirable to provide the support and rigidity of a conventional cast when immobilizing a part of the patient. Hence there remains the long sought-after need to provide a cast with the rigidity of a conventional cast that permits health care providers to inspect, treat and comfort the part of a patient within a cast without removal of the cast.

### SUMMARY OF INVENTION

It is an object of the present invention to provide a cast which is inherently more comfortable to the patient than a typical cast, and one which provides the patient with greater flexibility including the ability to immerse the casted portion of the patient in water.

It is also an object of the present invention to provide a cast which permits examination and application of a wide variety of treatments to the part of the patient's body disposed within the cast without removing the cast.

Further, it is an object of the present invention to provide a cast which permits the application a wide variety of treatments without significantly increasing the weight of the cast, the cost of the cast, or significantly increasing the difficulty in applying the cast to the patient or removing the cast from the patient.

It is an object of the present invention to provide a cast which lowers the pathogen bio load on the patient's skin through passive sterilization, and one which provides the patient with reduced risk of infection, itching and odor.

It is an object of the present invention to provide a cast capable of circulating a sterilizing fluid, such as ozone gas or ozonated water, to make contact with the part of a patient immobilized within the cast. It is also an object of the present invention to provide a reliable source of ozone and ozonated water for use with a cast constructed according to the present invention.

In accordance with the present invention, the part of the patient to be immobilized is placed within a stockinette that has a flexible, fluid impermeable shell that surrounds the part of the patient being treated and forms a continuous hollow sleeve that is open on at least one end. The shell is larger in cross section than the part of the patient being casted, thus forming a gap between the patient and the shell. The shell is constructed of a bendable sheet that is impermeable to fluids including air, ozone, water, ozonated water and many other fluids useful in treating or comforting a patient. Also, the sheet is tensionally rigid for the forces reasonably expected to be impressed upon the cast. The shell is constructed of light weight materials and is structurally unstable unless supported, the necessary support being provided by wrapping the shell with casting material which when cured forms a rigid structure.

The stockinette also has a flexible membrane disposed between the shell and the casted part of the patient, and the membrane is secured and sealed at its edges to the shell thereby forming with the shell a bag which surrounds the casted part of the patient. The membrane is constructed of flexible and stretchable plastic that is also impermeable to fluids that are useful in the treatment or care of patients, such as water, air, ozone and ozonated water. Since the membrane will be in contact with the patient, it is preferably constructed of non-allergenic plastic material that is soft to the touch. The shell has a sufficiently large cross section that the air tight bag, when deflated, is spaced from the portion of the patient within the cast by a gap. The gap is available to provide access to the part of the patient's body within the cast for examination, treatment, application of medications, sterilization, and comforting processes such as heating, cooling, bathing and drying.

The water and air impermeable bag is inflatable, and when inflated extends into and occupies the gap, thus engaging the body part of the patient being treated. At the discretion of the attending physician, the inflatable bag is capable of exerting a range of pressures between the body part of the patient and the shell, but the force produced by the inflatable bag is impressed as a tensional force on the shell. The shell requires support from the surrounding mass of casting material to withstand the tensional forces from the inflated bag. The inflatable bag assures a good fit between the body part and the cast, and may be evacuated to open the gap and provide access to the part of the patient disposed within the cast for observation, treatment, sterilization, temperature adjustment, or cleaning.

In accordance with the present invention, fluids may be circulated through a cast without removing the cast from the patient. With the inflatable bag of the cast deflated, the stockinette now serves as a conduit to flow fluid through the cast and in contact with the immobilized part of the patient. The flow of fluid through the cast may simply be for the comfort of the patient, such as flowing cool or warm dry air through the cast. The flow of fluid may also be a liquid to clean a wound, or a fluid to sterilize a wound. Ozone has been found to be a powerful sterilizing agent and the flow of ozone gas or ozonated water through the stockinette in contact with the immobilized part of a patient can be used to sterilize and clean a wound. It may also be desirable to repeat the sterilization process at intervals, and to be able to do so without removing the cast from the patient is an advantage of the present invention.

The present invention contemplates a light weight stockinette which covers the part of a patient to be immobilized within a cast and forms a wall between the mass of casting material and the portion of the patient being immobilized, that wall being spaced from the patient by a gap of ¼ to ⅝ inch. The wall formed by the stockinette provides a base upon which casting material, generally in the form of Plaster of Paris or fiberglass tape, may be applied. The stockinette also forms a fluid impermeable wall between the casted part of a patient and the casting material to permit the patient to be treated with a flow of fluid without leakage of the fluid into the casting material or the surrounding environment. The stockinette also has a bag on its inner surface directly confronting the casted part of a patient which may be inflated to expanded into and occupy the gap, thus engaging the casted part of the patient. Also, an inflated bag may be deflated to reduce the pressure on the casted part of the patient or to contract the bag to open the gap and expose the casted part of the patient for examination or treatment.

### DESCRIPTION OF THE DRAWINGS

The invention will be more fully described with reference to the accompanying drawings, in which:

FIG. 1 is an isometric view of a cast constructed according to the present invention disposed upon a limb of a patient:

FIG. 2 is a sectional view of the cast taken along the line 2-2 of FIG. 1;

FIG. 3 is a plan view of the inner surface of the shell of the cast of FIGS. 1 and 2 early in the process of constructing the cast according to the present invention;

FIG. 4 is an isometric view of a cast for use on a limb of a patient that constitutes another embodiment of the present invention, the stockinette being a substitute for the stockinette of the embodiment of FIGS. 1 through 3;

FIG. 5 is a plan view of the inner surface of the shell of the cast of FIG. 4 early in the process of constructing the stockinette of the second embodiment of the present invention;

FIG. 6 is a front elevational view of a stockinette as applied on a patient's foot, the patient's body parts and casting material being omitted for clarity;

FIG. 7 is a plan view of the outer surface of the shell of the stockinette of FIG. 6;

FIG. 8 is a front elevational view, partly in section, of a cast applied to a patient's leg and foot using the stockinette of FIGS. 6 and 7 and additional apparatus for fluid sterilization of the foot, some elements being diagrammatically illustrated; and

FIG. 9 is a fragmentary isometric view of metal particles imbedded onto the plastic membrane of FIGS. 6 and 7.

### DESCRIPTION OF THE PREFERRED EMBODIMENT

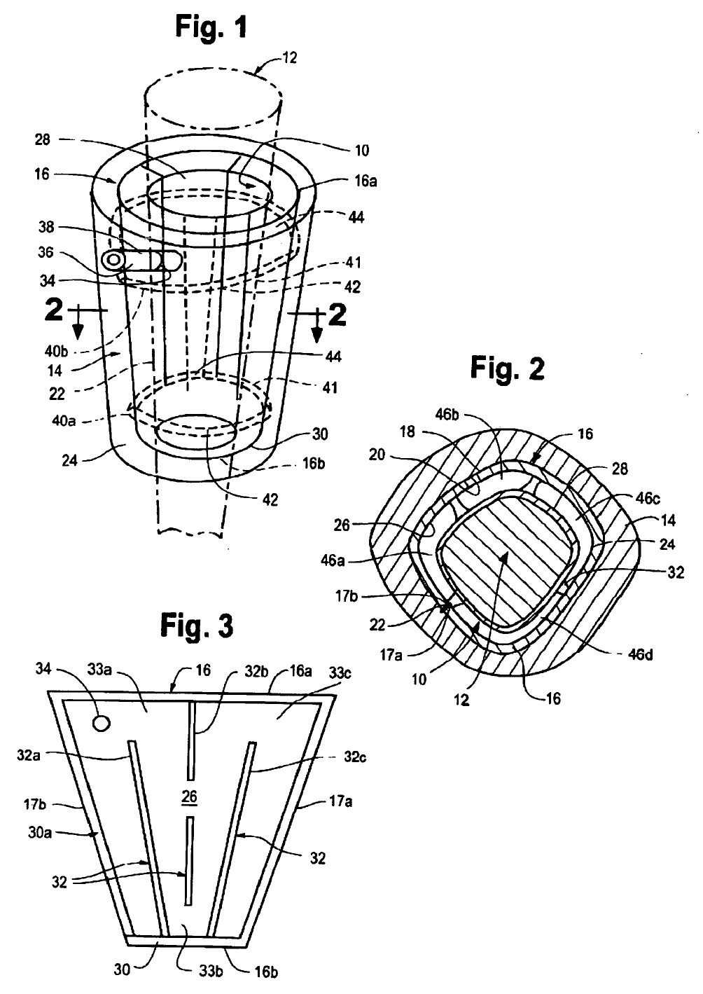

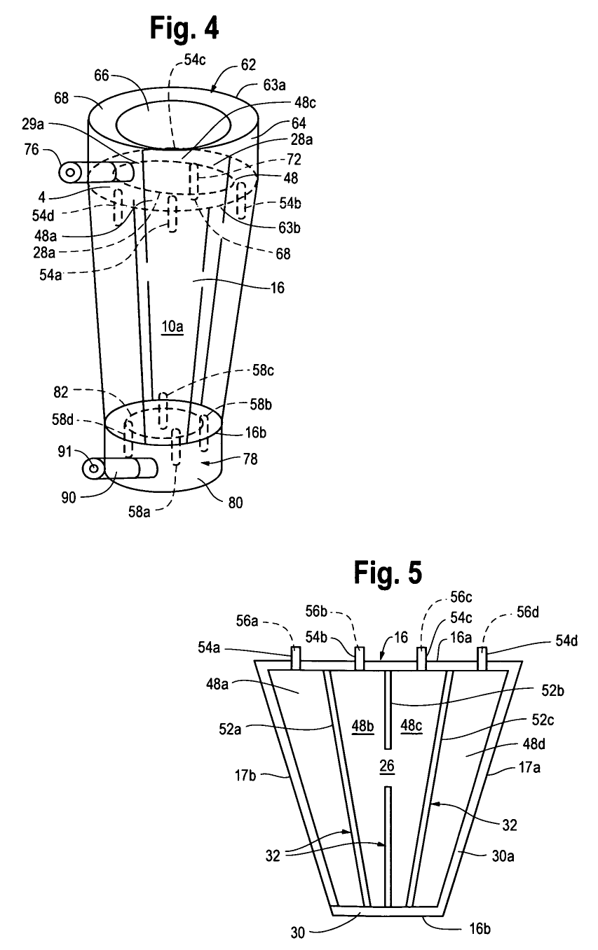

Like all orthopedic casts, a cast constructed according to the present invention has a shape adapted to accommodate the part of the patient being immobilized. For example, an inflatable stockinette for forming a short leg, full leg, forearm or full arm cast has the basic shape of a truncated cone and is open on at least one end and generally both ends. A cast of this type is illustrated in the embodiment of FIGS. 1 through 3, and the embodiment of FIGS. 4 and 5.

In FIGS. 1 and 2, a stockinette 10 is disposed about the immobilized part 12 of a patient, such as a limb, and a mass 14 of casting material, such as Plaster of Paris or fiberglass, is disposed about the stockinette. The stockinette 10 has a shell 16 in the shape of a truncated cone which has an outer surface 18 which engages an inner surface 20 of the mass 14 of casting material. The shell 16 is constructed of a relatively thin, flexible, fluid impermeable, solid sheet material which is transparent to roentgen radiation and non-allergenic. The shell 16 is preferably formed from a flat plastic sheet which has the approximate shape of a truncated triangle with generally parallel ends 16a and 16b and straight spaced edges 17a and 17b disposed at an acute angle, thus permitting the sheet to be wrapped around the part of a patient to be casted with edges 17a and 17b abutting each other and sealed and secured to each other as by thermal welding or cementing, thus forming a seam 22 along the axis of the abutting edges 17a and 17b. The shell 16 is constructed of material that has the flexibility to bend sufficiently to bring the edges 17a and 17b into abutment without stretching the shell beyond its elastic limit, and hence the shell assumes a generally truncated conical shape after bending. Not only is the shell preferably constructed of flexible material to permit the necessary bending, but it must also be constructed of material of sufficient tensional strength that the shell is able to transfer forces generated by the patient to the mass 14 of casting material. The shell is also preferably constructed of material having a high ratio of tensional strength to weight in order to produce a cast of light weight for the comfort of the patient.

The outer surface 18 of the shell 16 is relatively rough in order to facilitate adhesion of the surface 18 with the inner surface 20 of the mass 14 of casting material. The mass 14 of casting material is generally produced by winding a plurality of turns of casting tape 24 about the outer surface 18 of the shell 16 to build up the necessary thickness of the mass 14 of casting material and thereafter permitting the casting material to cure. The thickness of the mass 14 of casting material, as measured along the radius from the center line of the cast, need not be as great as it would be without the stockinette 10 of the present invention due to the tensional strength of the shell 16. The shell 16 may be constructed of a wide variety of materials, but a plastic material is generally preferable because of the flexibility, tensional strength and weight to tensional strength ratio of certain plastics. In a preferred construction of a cast according to the present invention, the shell 16 is constructed of 1/32^{nd} inch polyurethane and the radial thickness of the mass 14 of casting material is 1.5 inches.

The diameter of the inner surface 26 of the shell 16 is selected to provide a gap 27 between the shell and the immobilized part 12 of the patient, and the stockinette 10 has an inflatable bag, designated 29 in FIG. 2, disposed in the gap between an inner surface 26 of the shell 16 and the part of the patient disposed within the cast. There are a number of different ways that this bag may be provided, such as mounting a pre-manufactured bag constructed of flexible, stretchable, air impermeable material on the inner surface 26 of the shell 16. However, the preferred construction uses a thin flexible, fluid impermeable, stretchable membrane 28 attached and sealed at the ends 16a and 16b and edges 17a and 17b to the inner surface 26 of the shell 16. In a preferred construction, the membrane 28 is attached and sealed to the shell 16 by a narrow seam 30 formed between the membrane and the inner surface 26 of the shell 16 that may be a strip 30a of adhesive cement disposed along the ends 16a and 16b and edges 17a and 17b between the membrane 28 and the inner surface 26 of the shell 16, as illustrated in FIG. 3. The membrane 28 is also mounted on the shell 16 by a plurality of seams or ribs 32 disposed along equally spaced axes disposed between the edges 17a and 17b, thus dividing the airtight inflatable bag formed by the membrane 28 and shell 16 into a plurality of cavities of about equal volume. FIG. 3 illustrates three strips 32a, 32b, and 32c of adhesive cement disposed on and forming the seams 32 for securing the membrane 28 on the shell 16. The strips 32a and 32c extend from the end 16b toward the end 16a of the shell 16, but leave openings 33a and 33c between the ends of the strips 32a and 32c and the strip 30 at the edge 16a. In like manner, the central strip 32b extends from the end 16a toward the end 16b but leaves an opening 33b between the end of the strip 32b and the end 16b. As an alternative, thermal welding may be used to form the seams between the membrane 28 and the shell 16, thus eliminating the adhesive strips and mounting the membrane 28 on the inner surface 26 of the shell 16 completely by thermal welding.

The shell 16 is provided with an aperture 34 adjacent to the end 16a, and a stem 36 is mounted and sealed at one end within the aperture 34 and extends outwardly through the mass 14 of casting material. An air valve 38 is mounted within the stem 36 and permits air to be pumped into the air impermeable bag formed by the shell 16 and membrane 28 and prevents air from flowing from the air impermeable bag. The air valve 38 may be mechanically released or removed from the stem 36, thus permitting air to escape or be removed from the air impermeable bag and causing the membrane 28 to retract from the casted part of the patient, thus opening the gap 27 between the membrane 28 and the casted part 12 of the patient to facilitate examination or treatment of the patient. The embodiment of the present invention illustrated in FIGS. 1 through 3 utilizes a single stem 36 and air valve 38 to inflate the bag 29 formed by the shell 16 and membrane 28, the air openings 33a, 33b and 33c permitting the air to pass between the membrane seams 32.

Stockinette 10 may be made by a manufacturing process and delivered to a patient's care provider as a flat article which is thereafter wrapped about the part 12 of the patient to be casted and the edges 17a and 17b sealed together. However, the cast may be more precisely fit to the patient if the cast is at least partially custom made to the patient. A custom made cast is preferably made by first determining the dimensions of a suitable cast to treat a particular part of a patient by measuring the circumference of the largest portion of the part of the patient to be casted and the circumference of the smallest part of the patient to be casted. From these circumference measurements, the diameters of the largest and smallest portions of the patient may be calculated by dividing by 3.14. The desired diameters of the inner surface 26 of the shell 16 at the locations that the largest part and smallest part of the patient will be positioned within the shell may then be calculated by adding to these diameters twice the thickness of the desired gap between the patient and the shell; and these distances will then be used to calculate the distances between the edges 17a and 17b at the locations that the largest and smallest parts of the patient will occupy within the cast by multiplying these distances by 3.14. The sheet that will become the shell 16 and the associated inflatable bag 29 formed by the shell 16 and membrane 28 may thus be custom made with a minimum discomfort to the patient before the care provider begins the process of applying the cast to the part of the patient that is to be immobilized. After the membrane 26 has been mounted on the shell 16, the stockinette is ready to be applied to the part of the patient to be immobilized. The shell 16 of the stockinette is wrapped about the part of the patient to be immobilized with the membrane 28 confronting the part of the patient being casted. Even though the restorative forces of the flexible shell 16 tend to maintain the shell in a bent shape, it is unlikely that the shell will assume a shape with a circular cross-section and more likely that it will assume an oval shape and cross-section. Hence, the shell 16 is preferably supported to maintain a truncated conical shape until the mass 14 of casting material is applied, and this support may be in the form of spacers placed between the part of the patient being immobilized and the membrane 28, or preferably positioning two rings 40a and 40b of different diameters about the outer surface of the shell 16, as illustrated in FIG. 1. Each of the rings 40a and 40b are constructed of two semi-circular parts 41 rotatably attached to each other at one end by a pivot pin 42 and releasably connected to each other at the other end by a latch 44, so that the rings may be installed on the shell 16 with minimum inconvenience to the patient. The rings 40a and 40b are installed on the shell 16 adjacent to its smallest end 16b and moved toward the largest end 16a of the shell until each ring tightly engages the shell 16. The rings 40a and 40b are selected to have different diameters so that the ring 40b engages the shell 16 adjacent to the larger end 16b and the smaller ring 40a engages the shell 16 near the smaller end 16b of the shell.

The rings 40a and 40b may be left in position and the mass 14 of casting material applied on the shell 16, preferably by winding casting tape 24 around the outer surface 18 of the shell 16 to build up the desired thickness of casting material. The casting material is preferably either Plaster of Paris or fiberglass and resin.

The membrane seams 32a, 32b and 32c effectively divide the inflatable bag 29 formed by the membrane 28 and shell 16 into four interconnected chambers designated 46a, 46b, 46c and 46d in FIG. 2. Hence, when air is pumped through the stem 36 and aperture 34 into the bag 29, the air will migrate into all four chambers 46a, 46b, 46c and 46d, and the chambers will assume the same pressures. The pressure between the membrane 28 and the shell may be increased under the direction of a healthcare professional to cause the membrane to engage the part of the patient within the cast and place pressure on that part of the patient. Movement of the patient's casted part is also restricted by the membrane and air pressure within the air bag 29, but since the chambers 46a, 46b, 46c and 46d are interconnected, the pressure in these chambers will rapidly equalize and the air pressure will retard only movements of a rapid nature.

FIG. 4 illustrates a stockinette 10a that constitutes another embodiment of the present invention and may be substituted for the stockinette 10 of FIGS. 1 through 3. Components of stockinette 10a that are identical to components of stockinette 10 are referred to by the same reference numerals herein and in the drawings. The stockinette 10a has a bag 29a with a plurality of independent air chambers 48a, 48b, 48c, and 48d which are disposed between the shell 16 and the casted part 12 of the patient. The bag 29a is formed by a membrane 28a which is mounted on the inner surface 26 of the shell 16 in a manner similar to the membrane 28 of the embodiment of FIGS. 1 through 3.

As in the embodiment illustrated in FIGS. 1 through 3, the diameter of the inner surface 26 of the shell 16 of the embodiment of FIG. 4 is selected to provide a gap 27 between the shell and the casted part of the patient, and the inflatable bag 29a is disposed in the gap 27. As in the embodiment of FIGS. 1 through 3, there are a number of different ways to provide the bag 29a, but the preferred construction uses a thin flexible, fluid impermeable, stretchable membrane 28a attached and sealed to the inner surface 26 of the shell 16 by a seam 30 disposed at the ends 16a and 16b and edges 17a and 17b, as illustrated in FIGS. 4 and 5. The membrane 28a is also mounted on the shell 16 along a plurality of seams 50a, 50b and 50c disposed on axes equally spaced between the edges 17a and 17b, thus dividing the bag 29a formed by the membrane 28a and shell 16 into independent air tight chambers 48a, 48b 48c and 48d of about equal volume. FIG. 5 illustrates a strip 30a of adhesive cement forming seam 30 and three strips 52a, 52b, and 52c of adhesive cement disposed on three axes to form the seams 50a, 50b, and 50c. The strips 52a, 52b and 52c intersect with the perimeteral strip 30 of adhesive cement at both ends 16a and 16b of the shell 16. As an alternative, the membrane 28a may be mounted on the inner surface 26 of the shell 16 completely by thermal welding.

Each of the air chambers 48a, 48b, 48c and 48d is provided with an aperture 53 in the membrane 28a adjacent to the end 16a of the shell 16, and stems 54a, 54b, 54c and 54d are sealed within the apertures 53 to provide air passages to the air chambers. The stems 54a, 54b, 54c and 54d extend from the membrane 28a generally parallel to the central axis of the shell 16, and each stem is provided with an air valve 56a, 56b, 56c or 56d mounted and sealed within the stem to control the flow of air. Each of the air valves 56a, 56b, 56c and 56d is a one-way flow valve that permits the flow of air from an air source or pump, not shown, into an air chamber 48a, 48b, 48c or 48d provided the pressure within the chamber is less than a predetermined amount below that of the air source.

In the embodiment of FIGS. 4 and 5, each of the air chambers 48a, 48b, 48c and 48d is provided with a second aperture 57a. 57b, 57c and 57d, respectively, and a second stem 58a, 58b, 58c or 58d is mounted and sealed within each of the second apertures. Each of the second stems 58a, 58b, 58c or 58d has a one-way valve 60a, 60b, 60c or 60d sealed within the stem which permits flow of air from the connected air chamber 48a, 48b, 48c or 48d into the air source provided the pressure in the air chamber is greater than the pressure in the air source by at least a predetermined amount. The second apertures 57a, 57b, 57c and 57d and the stems 58a, 58b, 58c and 58d could be located at any location on their respective air chambers, but the stems are preferably mounted on the membrane 28a generally parallel with the central axis of the shell 16 and adjacent to the end 16b of the shell 16.

To adjust the air pressure in the independent air chambers 48a, 48b, 48c and 48d by connecting a single air source sequentially to each of the valve stems 54a, 54b, 54c and 54d is time consuming and may result in unacceptably unequal pressures in the air chambers. Accordingly, a cast constructed according to this embodiment of the invention may advantageously have a manifold 62 mounted on end 16a of the shell 16. The manifold 62 has an outer rectangular wall 64 with one edge 63a mounted on the end 16a of the shell 16, the outer wall 64 extending outward from the end 16a as an extension of the shell 16. The manifold 62 also has a rectangular inner wall 66 disposed spaced from and confronting the outer wall 64 and forming an airtight enclosure with the outer wall 64 and a pair of side walls 68a and 68b. The first of the side walls 68a is mounted and sealed on the edge 63a of the outer wall 64 and extends to and is sealed on the corresponding edge 69a of the inner wall 66. The outer wall 64 also has a second edge 63b disposed opposite the edge 63a and the second side wall 70 extends between the edge 63b and the corresponding edge 69b of the inner wall 66. When the stockinette 10a is applied to the patient, outer wall 64 and inner wall 66 of the manifold 62 are bent to form cylindrical structures and the opposite ends of each of the walls are sealed together.

As illustrated in FIG. 4, the valve stems 54a, 54b, 54c, and 54d are sealed within openings 72 in the first side wall 68 at the inner surface 26 of the shell 16. The inner wall 66 has a longitudinal axis shorter than the longitudinal axis of the outer wall 64. and after being bent to a generally cylindrical form has a diameter at least ½″ greater than the confronting part of the patient disposed within the cast in order to provide a continuation of the gap 27 through the manifold 62 for purposes of providing access to the patient's part within the cast. A hollow tubular air connector 74 extends outwardly from the outer wall 64 of the manifold 62, and the connector is provided with a one-way valve 76 that permits air to enter the manifold providing the air source has a pressure exceeding a predetermined pressure above the pressure in the manifold, thereby connecting each of the air chambers 48a, 48b, 48c and 48d to a single source of compressed air. The air pressure within the manifold 62 can be reduced by loosening or removing the one-way valve 76 from the air connector 74, and the pressure in the manifold 62 can be adjusted to and maintained at a pressure up to the lowest pressure required to initiate a flow of air into one of the air chambers 48a, 48b, 48c or 48d.

Depending on the materials used in the manifold 62, the manifold may function solely to control the flow of air from an air source, such as a pump, to the air chambers 48a, 48b, 48c and 48d, or perform this function and provide an end closure about the part of the patient being immobilized. If the manifold is solely for the purpose of controlling the flow of air into the air chambers, the walls 64 and 66 and the sides 68 and 70 are constructed of sheet material of limited flexibility, such as the plastic material of the shell 16. In the preferred embodiment, the manifold is constructed to function as an end closure for the cast, and the outer wall 64 is constructed of air impermeable material of limited flexibility similar to the shell 16, but the inner wall 66 and the side walls 68 and 70 are constructed of air impermeable flexible and stretchable material similar to the membrane 28a. With these materials, the manifold 62 may be maintained at a pressure up to the pressure of the air chambers and expand into the gap 27 between the outer wall 64 and the confronting part of the patient.

In like manner, a second manifold 78 is mounted on end 16b of the shell 16, and the open end of each second stem 58a, 58b, 58c or 58d of the independent air chambers 48a, 48b, 48c, and 48d communicates with the second manifold 78. The second manifold 78 is constructed similarly to the first manifold 62 and has an outer wall 80 and an inner wall 82 mounted in spaced relation to each other by a first side wall 84 and a second side wall 86 that extend between opposite sides of the walls 80 and 82. As illustrated in FIG. 4, the first side wall 84 of the manifold 78 is mounted on the end 16b of the shell 16 and extends there from. The open ends of the valve stems 58a, 58b, 58c, and 58d are sealed within openings 88 in the first side wall 84 at the inner surface 26 of the shell 16.

Like manifold 62, the inner wall 82 has a longitudinal axis shorter than the longitudinal axis of the outer wall 80. and after being bent to a generally cylindrical form has a diameter at the least ½″ greater than the confronting part of the patient disposed within the cast in order to provide a continuation of the gap 27 through the second manifold 78 for purposes of providing access to the patient's part within the cast. A hollow tubular air connector 88 extends outwardly from the outer wall 80 of the manifold 78, and the connector is provided with a one-way valve 90 that permits air to exit the manifold providing the air pressure in the manifold is greater than the pressure at the inlet of the air connector by at least a threshold pressure. The air pressure within the second manifold 78 can be maintained by plugging into the air connector 74, and the manifold 78 can be adjusted to approach and maintain a pressure up to the highest pressure within the air chambers 48a, 48b, 48c or 48d.

Depending on the materials used in the second manifold 78, the manifold may function solely to control the flow of air from the air chambers 48a, 48b, 48c and 48d, or perform this function and provide an end closure between the cast and the part of the patient being immobilized. If the manifold is solely for the purpose of controlling the flow of air from the air chambers, the walls 80 and 82 and the sides 84 and 86 are constructed of sheet material of limited flexibility, such as the plastic material of the shell 16. In the preferred embodiment, the second manifold 78 is constructed to function as an end closure for the cast, and the outer wall 80 is constructed of air impermeable material of limited flexibility similar to the shell 16, but the inner wall 82 and the side walls 84 and 86 are constructed of air impermeable flexible and stretchable material similar to the membrane 28a. With these materials, the second manifold 78 may be maintained at a pressure approaching the pressure of the air chambers 48a, 48b, 48c and 48d and expand into the gap 27 between the outer wall 80 and the confronting part of the patient.

As in the embodiment of FIGS. 1 through 3, the stockinette 10a is first assembled in flat configuration with the membrane 28a on the shell 16 and the manifolds 62 and 78 mounted on the ends 16a and 16b if the shell, respectively. The stockinette 10a is then ready to be applied to the part of the patient to be immobilized. The assembled stockinette is then bent around the part of the patient to be immobilized with the inner surface 26 of the shell 16 and the membrane 28a confronting the part of the patient being casted, and the edges 17a and 17b and the ends of the out wall 80, inner wall 82, first side wall 84 and second side wall 86 are brought into contact, respectively. Thereafter, the edges 17a and 17b, the ends of the outer wall 80, the ends of the inner wall 82, the ends of the first side wall 84 and the ends of the second side wall 86 are connected and sealed together as by cementing or thermal welding. Then, the shell 16 is supported to maintain a truncated conical shape and a mass 14 of casting material is applied. The membrane seams 50a, 50b and 50c effectively divide the bag 29a formed by the membrane 28a and shell 16 into four independent air chambers 48a, 48b, 48c and 48d. Each of these air chambers 48a, 48b, 48c and 48d is then inflated to expand the air chambers into abutment with the casted part of the patient by pumping air through the stems 54a, 54b, 54c and 54d. The pressure between the membrane 28a and the patient may be increased by increasing the air pressure in the chambers 48a, 48b, 48c and 48d under the direction of a healthcare professional. Movement of the patient's casted part will be directed toward one or more of the air chambers and away from one or more of the other air chambers. Since the air chambers are independent, the body of air within each of the chambers cannot migrate from one chamber to another, and thus the air chambers subject to the force of the patient's movement increase in pressure and resist movement of the patient's casted part. Accordingly, casts constructed in the manner of the embodiment of FIGS. 4 and 5 are significantly stiffer than the casts of the embodiment of FIGS. 1 through 3 under the same conditions and air pressure.

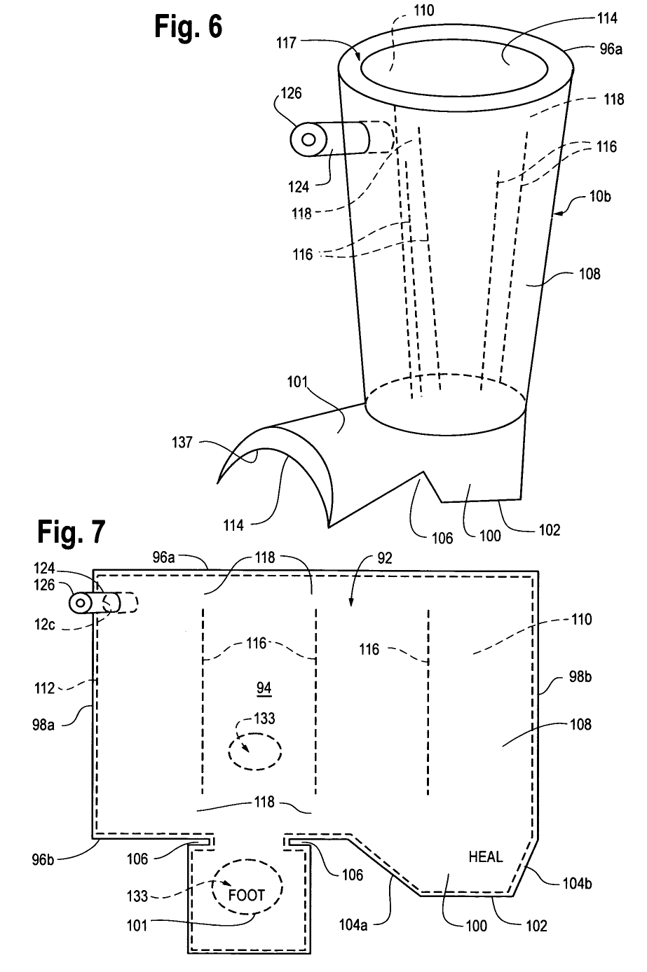

Some body parts, such as a hand or a foot, may be immobilized by applying a cast that constitutes another embodiment of the present invention to a part of the patient adjacent to the body part to be immobilized and to the part to be immobilized. FIGS. 6 through 8 illustrate a cast constructed in this manor for immobilizing a foot. A cast constructed according to the present invention may be applied to a foot to facilitate healing that is not designed to permit the patient to walk, or the cast may be applied to the patient with a construction permitting the patient to walk while wearing the cast. In the embodiment of FIGS. 6 through 8, the cast is intended to facilitate treatment of the foot, such as is required by an open wound or diabetic ulcer on the foot, and it is not contemplated that the patient will walk while the cast is on the patient's foot. A walking cast constructed according to the present invention can be applied to a patient's foot by incorporating a conventional walking cast support structure in a cast using a stockinette according to the present invention.

As in the embodiments of FIGS. 1 through 5, the cast of the embodiment of FIGS. 6 through 8 has a stockinette 10b and a mass of casting material 14 in the form of tape 24 wound about the stockinette. However, the stockinette 10b of FIGS. 6 through 8 has a shell 92 with a first portion 94 with opposite ends 96a and 96b and opposing sides 98a and 98b, similar to the shells 10 and 10a of FIGS. 1 through 5. The shell 92 also has two additional portions extending outwardly from the end 96b, namely, a heel appendage 100 and a foot appendage 101, as illustrated in FIG. 7. The heel appendage 100 is located on the end 96b adjacent to the side 98b of the shell 92, and it has an end 102 parallel to the end 96b of the first part of the shell 92 and tapered edges 104a and 104b extending between the opposed ends of the end 102 and the end 96b of the shell 92. The foot appendage 101 also extends from the end 96b of the shell 92 between the heel appendage 100 and the side 98a of the shell 92. The foot appendage 101 is rectangular in shape and has a pair of elongated slots 106 extending therein from opposite sides and on and parallel to the end 96b of the shell 92.

FIG. 7 is a plan view of the outer surface 108 of shell 92. The opposed inner side 110 of the shell 92 has a strip 112 of adhesive cement that is disposed adjacent to the sides 98a and 98b, the ends 96a and 96b, and the edges of the heel appendage 100 and foot appendage 101 to form a continuous perimeteral strip. A membrane 114 that corresponds to the membranes 28 and 28a of the embodiments of FIGS. 1 through 5 confronts the entire inner surface 110 of the shell 92 and is mounted on and sealed to the shell 92 by the strip 112. Also on the inner surface 110 of the shell 92, three linear strips 116 of adhesive cement are disposed on equally spaced linear axes parallel to and between the sides 98a and 98b of the shell 92 to support the membrane 114 on the shell 92, thereby forming an inflatable expandable bag 117. The strips 116 terminate at each end at a distance from the strip 112, thereby leaving gaps 118 for the flow of air throughout the space between the membrane 114 and the inner surface 110 of the shell 92. Like the membranes 28 and 28a, the membrane 114 is constructed of elastic, stretchable fluid impermeable material, preferably thin plastic sheet. In a preferred construction, the membrane 114 is constructed of a sheet of polyurethane with a thickness of 1/64 inch. The shell 92, like the shells 16, is constructed of bendable fluid impermeable sheet with sufficient tensional strength to withstand the forces that a patient is likely to exert on the mass of casting material employed in the cast. In the preferred construction, the shell 92 is constructed of polyurethane with a thickness of 1/32 inch.

An aperture 120 is disposed in the shell 92 near the intersection of the end 96a and the side 98a, and a hollow stem 124 is sealed within the aperture 120, thereby providing a means for transporting air into the expandable bag 117 formed by the shell 92 and membrane 114. A one-way valve 126 is removably mounted within the stem 124 to permit air to be pumped into the bag 117 and to prevent air from flowing out of the bag through the stem 124.

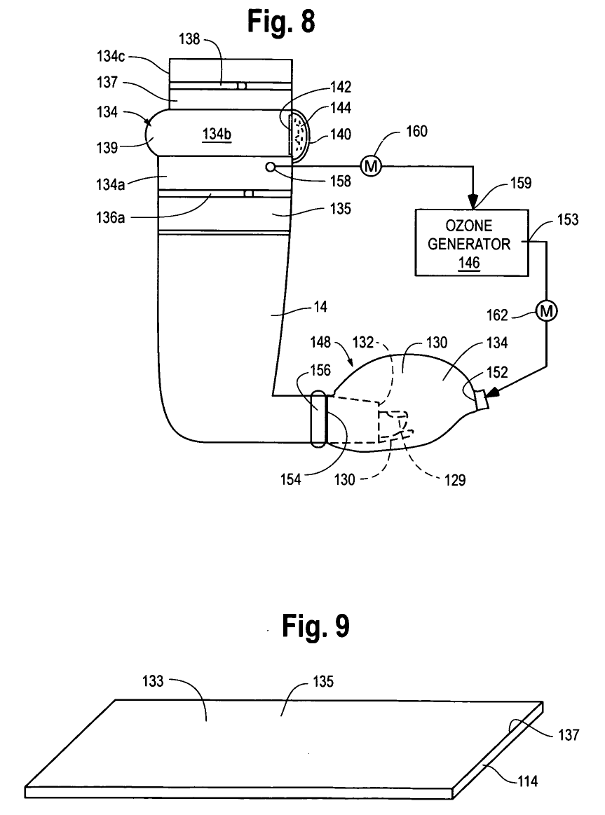

The assembly of shell 92, membrane 114 and stem 124 may be prepared and acquired from a laboratory as a commercial product or prepared by the attending health care providers immediately prior to applying the cast to a patient. Generally, the patient's leg and foot are prepared for casting by wrapping the foot and leg in cotton padding and placing the wrapped foot and leg in a cotton stockinette. The first step in applying the cast to a patient is to bend the foot appendage 101 approximately 90 degrees outwardly from the outer surface 108 of the shell 92 to provide clearance for shaping the assembled shell 92 and bag 117 on the patient. The patient's foot and lower leg are now placed over the bag 117 and inner surface 110 of the shell 92 with the patient's heel disposed centrally of the heel appendage 100 and the bottom of the patient's foot is positioned to confront the end 102 of the heel appendage 100. The assembled shell 92 and bag 117 are then bent around the patient's heel. Thereafter, the assembled shell 92 and bag 117 are bent around the remainder of the patient's lower leg, and the opposing sides 98a and 98b of the shell are brought into abutment. The abutting sides 98a and 98b are thereafter secured and sealed together forming a seam 128, as by adhesive cement or thermal welding. Next, the health care professional will place a foot support 130 below the patient's sole, and a mass of casting material is applied to the outer surface 108 of the shell 92 while maintaining the shell in the desired shape as described in the embodiments of FIGS. 1 through 5. It is generally desirable to inflate the bag 117 sufficiently to provide the desired spacing between the foot of the patient and the cast before applying casting material. The casting material is most conveniently applied by winding casting tape about the patient's foot and outer surface 108 of the shell 92, but the patient's toes illustrated at 129 of FIG. 8 are left out of the cast and there is an opening 132 into the gap between the shell 92 and the patient's immobilized parts.

After the casting material has fully cured, the bag 117 may be inflated to provide a uniform pressure on the patient's lower leg and foot as determined by the attending health care professional. The cast will be as comfortable for the patient as the patient's condition permits since it delivers uniform pressure without high pressure areas. In addition, the bag 117 can be deflated at any time for cooling the patient's immobilized parts with a flow of cool air through the cast or heating with a flow of warm air through the cast. Further, the patient's comfort can be improved by cleaning or soothing with liquids applied under the cast.

It is a great advantage of the invention that the patient's immobilized part can be observed and treated by the attending health care professional without removing the cast. With the bag 117 deflated, a wound can be observed from outside of the cast by inserting an endoscope in the gap between the deflated bag 117 and the patient. Also, medication may be applied to a wound by inserting an instrument into the gap formed by deflating the bag 117.

The warm moist atmosphere that exists when a body part has been confined within a cast for a significant time is favorable for the growth of pathogens that are likely to infect a wound. The most common of these pathogens are Coryne bacterium spp., Enterococcus faecalis, Klebsiella pneumoniae, Kockuria Kristinae, Morganella Morganii, Peptoniphilus, Pseudomonas aeruginosa, Staphylococcus aureus and Steptococcus pneumoniae. Growth of these microorganisms and many other microorganisms may be significantly retarded by use of the oligodynamic properties of certain metal particles. Small particles in significant concentrations of silver, copper, aluminum, titanium, carbon nanoparticles, palladium, platinum, brass and bronze have been found to be effective in reducing the population of the pathogens that cause infections in wounds identified above. By applying the oligodynamic principle to the cast of FIGS. 6 and 7, the inventors have provided a self sterilizing cast particularly suited for treatment of wounds of the legs and feet, and particularly diabetic foot ulcers.

As illustrated in FIGS. 6, 7 and 9, a concentration 133 of small metal particles 135 is secured on the outer surface 137 of the membrane 114 positioned to confront the foot of a patient, and particularly the wound on the foot of a patient. The metal particles are of silver, copper, aluminum, titanium, carbon nanoparticles, palladium, platinum, brass or bronze, and each particle has a cross section no greater than 0.02inch and the mean distance between particles is approximately 0.04 inch. In a preferred construction, the particles are carbon nanoparticles. Depending on the patient, the self sterilizing cast may provide sufficient deterrent to pathogens that the periodic observation and treatment capability of the cast is sufficient protection against infection, itching and odor. If desirable, the self sanitizing cast may be used in combination with the gas sterilization process described in FIG. 8 and hereafter or that process may be used without the metal particles 135.

FIG. 8 illustrates a process for sterilizing a wound or diabetic ulcer on the foot of a patient that has been immobilized by application of the cast illustrated in FIGS. 6 through 8. First, the valve 126 is removed from the stem 124 and the bag 117 is fully deflated to open the gap between the inner surface 110 of the shell 92 and the leg and foot of the patient. An upper sleeve assembly 134 is then slipped over the patient's foot and positioned at the interface of the end 96a of the shell 92 of the cast and the patient's leg.

The upper sleeve assembly 134 is a hollow plastic structure having three interconnected regions 134a, 134b and 134c. The first region 134a has a circular band 135 of relatively heavy flexible plastic that encompasses the layer of casting tape at the upper end of the cast. A belt 136 of self locking material such as Velcro surrounds the central portion of the band 135, and the belt is tightened on the band 135 to secure the first region of the upper sleeve assembly on the upper end of the cast and to provide a gas tight seal between the cast and the sleeve assembly.

The third region 134c of the upper sleeve assembly 134 is constructed similar to the first region 134a, but the third region surrounds and forms a fluid tight seal about the patient's leg. The third region 134c has a circular band 137 of relatively heavy flexible plastic that encompasses the patient's leg, and a belt 138 of self locking material such as Velcro surrounds the central portion of the band 137. The belt 138 is tightened to secure the third region 134c of the upper sleeve assembly 134 on patient's leg and to provide a gas tight seal between the upper sleeve assembly 134 and the patient.

The second region 134b of the upper sleeve assembly 134 connects the first region 134a to the third region 134c. The second region 134b of the upper sleeve assembly 134 has an outwardly extending circular band 139 of stretchable gas permeable flexible plastic that loosely surrounds the patient's leg. A circular chamber 140 is formed between the band 139 and a gas permeable ring 142 mounted on the inner surface of the circular band 139, and a mass 144 of carbon granules, such as activated charcoal, is disposed within the circular chamber 140 of the circular band 139.

Most sterilizing gases can be used to sterilize the cast illustrated in FIGS. 6 through 8. Ozone is a sterilizing gas that is recognized to be particularly effective, and is therefore preferred in this invention. After applying the upper sleeve assembly 134 to the patient, an ozone generator 146 is connected to the gap between the shell and the parts of the patient within the cast by a foot coupler 148. The foot coupler 148 has a soft plastic sleeve 150 with an inlet 152 connected to the ozone outlet 153 of the ozone generator 146. The sleeve 150 also has an opening 154 which is slipped over the toes of the patient to surround the portion of the cast that encloses the large portion of the foot of the patient. A belt 156 encircles the sleeve 150 adjacent to the opening 154 and secures the sleeve on the cast and provides a gas tight seal to the cast.

In operation, ozone from the generator 146 flows through the foot coupler 148 into the gap between the shell 92 of the cast and the patient's immobilized parts. The flow of ozone gas continues throughout the entire gap between the patient and the shell 92 of the cast, and thereafter passes through the first portion 134a of the upper sleeve assembly 134. Thereafter, the ozone gas flows into the second portion 134b of the upper sleeve assembly, penetrates the ring 142 of the second portion 134b of the upper sleeve assembly and passes through the carbon granules 144. The carbon granules 144 react with the ozone to reduce the ozone to oxygen which passes through the porous band 139 into the ambient atmosphere. The carbon granules 144 act as an ozone destroyer to protect the surroundings and people present from the effects of ozone.

The ozone generator 146 uses oxygen as a starting material for production of ozone, and the spent ozone disposed within the upper sleeve assembly 134 may be advantageously used as a source material for the ozone generator. Accordingly, a stem 158 is inserted through and sealed to the plastic belt 135 to vent spent ozone, and the stem is connected to the ozone inlet 159 of the ozone generator 146. Ozone is unstable and decays into oxygen within a time period of minutes, and therefore it is desirable to measure the ozone content of the gas that passes out of the cast. Hence a meter 160 that measures the percent of ozone in the gas flow is inserted in the connecting tubing between the upper sleeve assembly 134 and the oxygen inlet 159 of the ozone generator 146. Also, it may take some time for an ozone generator to achieve efficient production of ozone following start-up. In order to assure the proper period of ozone sterilization, a meter 162 that measures the percent of oxygen in the gas flow is also provided in the connection between the ozone output 153 of the ozone generator 146 and the foot coupler 148 to measure the ozone entering the foot coupler.

Those skilled in the art will apply modifications, recognize applications and devise uses for the present invention in addition to those set forth in this specification. It is therefore intended that this invention be not limited by the foregoing disclosure, but only by the appended claims.

The invention claimed is:

1. A cast for treatment of a part of a human or animal body that is likely to benefit from being immobilized comprising a bendable shell that is adapted to surround the portion of the body to be treated, said shell having an inner surface that is adapted to confront the part of the body to be casted and an opposed outer surface, said shell also having opposed linear edges and opposed ends extending between the edges, said shell being adapted to have a bend forming the shell into a configuration with opposed edges abutting each other, means disposed on the shell to secure and seal said opposed edges to each other, said shell being adapted to surround the part of the body to be treated with the part of the body extending from at least one end of the shell and the inner surface of the shell being spaced from the body part of the patient, means operatively associated with the shell forming an inflatable and expandable bag on the inner surface of the shell, said bag being adapted to expand upon being inflated and engage the part of the patient disposed within the shell and said bag being adapted to contract upon being deflated to form a gap between the bag and the part of the patient disposed within the shell of at least 0.25 inch, valve means operatively associated with the bag for connecting a pressurized source of fluid to the bag and controlling the pressure of the fluid within the bag, and a mass of casting material applied to and about the outer surface of the shell to maintain the shell in a fixed shape, said shell having sufficient tensional strength to maintain its shape during application of the casting material.

2. A cast for treatment of a part of the body of a patient that is likely to benefit from being immobilized comprising claim 1 wherein the shell is constructed of air impermeable material and the means forming an inflatable and expandable bag comprises a fluid impermeable flexible and stretchable membrane with a perimeter, said membrane being mounted and sealed at its perimeter on the inner surface of the shell to form a fluid tight chamber between the inner surface of the shell and the membrane, said membrane being adapted to stretch in response to an increase in fluid pressure in the chamber.

3. A cast for treatment of a part of the body of a patient that is likely to benefit from being immobilized comprising claim 2 wherein a plurality of generally straight parallel seams disposed between the inner surface of the shell and the confronting surface of the membrane secure and seal the membrane on the shell and divide the chamber into a plurality of cavities, each of the cavities communicating with the valve means for connection to a fluid source of pressure.

4. A cast for treatment of a part of the body of a patient that is likely to benefit from being immobilized comprising claim 3 wherein each seam between cavities is provided with an aperture in the seam, thereby connecting each cavity for fluid flow with adjacent cavities.

5. A cast for treatment of a part of the body of a patient that is likely to benefit from being immobilized comprising claim 3 wherein each cavity is sealed from every other cavity, and each cavity is connected to the valve means for fluid inflation and pressure control.

6. A cast for treatment of a part of the body of a patient that is likely to benefit from being immobilized comprising claim 1 wherein a plurality of particles selected from one or more members of the group silver, copper, aluminum. Titanium, carbon nanoparticles, palladium, platinum, brass and bronze is disposed on the surface of the inflatable bag opposite the shell and adapted to confront the body part of a patient.

7. A cast for treatment of a part of the body of a patient that is likely to benefit from being immobilized comprising claim 1 wherein the opposed linear edges of the shell are of equal length and the opposed ends of the shell are of substantially different lengths, whereby the shell assumes a generally truncated connical configuration when the shell is bent and the linear edges abut each other.

8. A cast for treatment of the leg and foot of a patient that is likely to benefit from being immobilized comprising claim 2 wherein the shell has an outwardly extending appendage from one of the ends thereof, said appendage being generally rectangular in shape and adapted to wrap over a patient's foot, and said appendage having a pair of slots extending therein from opposite sides and aligned with the adjacent end of the shell, and the membrane having an outwardly extending portion confronting the appendage and mounted and sealed at its perimeter on the appendage.

9. A cast for treatment of the leg and foot of a patient that is likely to benefit from being immobilized comprising claim 8 wherein a plurality of metal particles selected from the group silver, copper, aluminum, titanium, carbon nanoparticles, palladium, platinum, brass and bronze are disposed on the surface of the outwardly extending portion of the membrane and adapted to confront the foot of a patient.

10. A cast for treatment of a body part of a patient that is likely to benefit from being immobilized comprising claim 2 wherein a plurality of metal particles selected from the group silver, copper, aluminum, titanium, carbon nanoparticles, palladium, platinum, brass and bronze are disposed on the surface of the membrane and adapted to confront the body part of a patient.
